(19) United States
(12) Patent Application Publication
(10) Pub. No.: US 2009/0306342 A1
(43) Pub. Date: Dec. 10, 2009

(54) BINDING OF MOLECULES

(75) Inventors: Joe Nobuyoshi Maeji, Wishart (AU); Alain-Dominique Jean-Pierre Gorse, Wishart (AU); Raisa Leonidovna Monteiro, Forest Lake (AU)

Correspondence Address: HOWREY LLP-CA, C/O IP DOCKETING DEPARTMENT, 2941 FAIRVIEW PARK DRIVE, SUITE 200, FALLS CHURCH, VA, 22042-2924, US

(73) Assignee: BIO-LAYER PTY LIMITED, Eight Mile Plains (AU)

(21) Appl. No.: 12/158,963

(22) PCT Filed: Dec. 29, 2006
(86) PCT No.: PCT/AU06/02010

§ 371 Date: Nov. 19, 2008

Related U.S. Application Data
(60) Provisional application No. 60/755,634, filed on Dec. 30, 2005.
(60) Provisional application No. 60/786,949, filed on Mar. 28, 2006.

(51) Int. Cl. C07K 1/14; C07H 21/00; C07K 16/00

(52) U.S. Cl. 530/344; 536/23.1; 530/387.1

(57) ABSTRACT

A method of binding a target molecule from an analyte containing the target molecule in admixture with at least one non-target molecule, which method comprises exposing the analyte to a substrate comprising first and second surface binding components, wherein the first surface binding component has specific binding affinity with respect to the target molecule but is independently unable to provide effective binding of the target molecule to the substrate, wherein the second surface binding component has non-specific binding affinity with respect to the target molecule and is independently unable to provide effective specific binding of the target molecule to the substrate, and wherein the target molecule is immobilized on the substrate by the combined binding effect of the first and second surface binding components.

### FIELD OF THE INVENTION

The present invention relates to a method for immobilizing a target molecule on a substrate. The invention also relates to reagents useful in this method. The invention further relates to a method of designing high affinity and high specificity “surface capture agents” useful for binding and detection of molecular species, such as biomolecular targets in diagnostics, drug discovery research, bio-separations and proteomics. The invention further relates to a method of modulating the binding efficacy to a target molecule of a structural feature that is present on a substrate surface (for example, peptide, small molecule, polynucleic acid ligands, etc) and that has some measurable affinity and specificity to the target molecule through extensions of essentially non-specific surface binding components.

### BACKGROUND OF THE INVENTION

In a number of technology fields there is a continuing need to identify high quality capture agents that are capable of binding specific molecules of interest with high affinity and specificity. For instance, in the development of proteomics and protein microarrays, the lack of protein-binding molecules as capture agents is one of the major factors for slower than expected progress. Protein microarrays for detection and functional studies of proteins require a capture agent for each and every target (protein) molecule and identifying the many thousands of different but specific capture agents that do not bind other proteins in complex mixtures is highly challenging. Such capture agents are usually biological molecules since high binding affinity and specificity are normally described in terms of three-dimensional shape complementarity with “recognition” being an outcome of a precise fit between surface regions of a target molecule and its complementary capture agent. For further discussion reference may be made to D. E. Koshland Jr., (1994), The key-lock theory and the induced fit theory, Angew. Chem. Int. Ed. Engl., 33: 2375-237 and C. Berger, S. Weber-Bornhauser, J. Eggenberger, S. Hanes, A. Pluckthun and H. R. Bosshard. (1999), Antigen recognition by conformational selection. FEBS Lett., 450: 149-153.

Antibodies are the most common of all capture agents but conventional routes of immunization to generate anti-sera are tedious, expensive and result in high failure rates. Alternative technologies to generate millions and, possibly, billions of antibodies through phage libraries, through use of alternative protein scaffolds such as human fibronectin and staphylococcus protein A are just some of the protein-based approaches being developed. Non-protein based capture agents such as nucleic acid aptamers are also used as they can be generated to virtually any class of target molecules via highly efficient in vitro combinatorial chemistry-type processes. Potential capture agents can also be readily identified from combinatorial libraries of peptides, peptidomimetics, small molecule compounds or any large collection of molecules, but they rarely bind with affinities approaching a good antibody and consequently are inadequate to capture target molecules that are present in low abundance in complex mixtures. To achieve higher binding affinity and specificities from such “leads” is highly challenging with no guarantee of success. Such ligands can also be used in affinity chromatography but, in this case, there is the extra challenge of removing the target molecule after affinity capture. If the binding is too strong, the target molecule can be damaged during release. However, a weak binding ligand is inadequate for efficient purification and finding the right balance can also be challenging.

The present invention seeks to provide an alternative approach whereby a target molecule can be immobilized or captured on a substrate without the need to identify an individual capture agent that has suitably high binding and specificity to the target molecule of interest. The present invention relies on a combination of independently ineffective binding interactions to bind a target molecule to a substrate. This approach is believed to afford significant flexibility with respect to design of a capture surface since it avoids the need to identify an individual capture agent having the characteristics described.

### SUMMARY OF THE INVENTION

Accordingly, in one aspect, the present invention provides a method of binding a target molecule from an analyte containing the target molecule in admixture with at least one non-target molecule, which method comprises exposing the analyte to a substrate comprising first and second surface binding components,

wherein the first surface binding component has specific binding affinity with respect to the target molecule but is independently unable to provide effective binding of the target molecule to the substrate,

wherein the second surface binding component has non-specific binding affinity with respect to the target molecule and is independently unable to provide effective specific binding of the target molecule to the substrate, and

wherein the target molecule is immobilized on the substrate by the combined binding effect of the first and second surface binding components.

It will be appreciated that binding of a target molecule in accordance with the present invention allows the target molecule to be extracted from the analyte.

In accordance with the present invention a target molecule of interest may be resolved from a mixture of molecules by binding/immobilization of the target molecule on a substrate, the surface characteristics of which have been designed to potentiate binding of the target molecule as opposed to one or more other molecules that are present in the mixture but that it is not desired to extract from the mixture. Herein these one or more other molecules are referred to as “non-target” molecules. In accordance with the present invention preferential and stable binding of the target molecule is achieved by the combined binding effect of surface components (first and second surface binding components) that are present on the substrate surface. Individually, each type of surface component is not effective in binding the target molecule at the prevailing conditions under which the substrate and the analyte are exposed to each other, irrespective of the density of the individual surface components on the substrate surface. However, the sum of the binding interactions between the surface components and the target molecule is sufficient to result in selective and stable binding of the target molecule, thereby allowing it to be isolated from a mixture.

In general terms, and in principle, the present invention is based on the premise that any two or more types of surface component present on the surface of a solid support could have the ability to function collectively as a capture agent for a target molecule of interest even though individual binding interactions between each type of surface component and the target molecule may be relatively weak and/or sufficiently non-specific to support selective and stable binding. This should be contrasted with prior art approaches where binding of a target molecule is reliant on the identification of individual molecules that have suitably high selectivity and binding efficacy to effect binding in their own right and the provision of such components on a substrate surface without detriment to the intended binding functionality of the component.

Looking at it another way, in accordance with the present invention the binding efficacy of a surface binding component that has specificity but insufficient binding strength with respect to a target molecule can be further strengthened and made practically effective by use of that surface binding component in combination with another suitably selected surface binding component that is non-specific to the target molecule but that has the effect (under the prevailing conditions) of modifying the overall association and/or dissociation rates with respect to binding of the target molecule and thus influence the final dissociation constant (Kd) associated with the binding event.

Accordingly, in another aspect, the present invention provides a method of increasing the binding effect of a substrate with respect to a target molecule, the substrate comprising a first surface binding component that has specific binding affinity with respect to the target molecule but that is unable to provide effective binding of the target molecule to the substrate, which method comprises providing on the substrate a second surface binding component that has non-specific binding affinity with respect to the target molecule and that is unable to provide effective specific binding of the target molecule to the substrate, such that the combined binding effect of the first and second surface binding components results in binding of the target molecule to the substrate.

In similar fashion, in another aspect, the present invention provides a method of increasing the binding effect of a substrate with respect to a target molecule, the substrate comprising a first surface binding component that has non-specific binding affinity with respect to the target molecule and that is unable to provide effective binding of the target molecule to the substrate, which method comprises providing on the substrate a second surface binding component that has specific binding affinity with respect to the target molecule but that is unable to provide effective specific binding of the target molecule to the substrate, such that the combined binding effect of the first and second surface binding components results in binding of the target molecule to the substrate.

It will be appreciated that the underlying principles associated with these embodiments of the invention are consistent with the principles explained above, and unless context otherwise permits the disclosure herein applies to both embodiments.

In another embodiment, the present invention provides a surface capture agent suitable for extracting a target molecule from an analyte, the surface capture agent comprising first and second surface binding components, wherein the first surface binding component has specific binding affinity with respect to the target molecule but is independently unable to provide effective binding of the target molecule to the substrate, wherein the second surface binding component has non-specific binding affinity with respect to the target molecule and is independently unable to provide effective specific binding of the target molecule to the substrate, and wherein the target molecule is immobilized on the substrate by the combined binding effect of the first and second surface binding components.

### DETAILED DISCUSSION OF THE PRESENT INVENTION

In accordance with the present invention binding of a target molecule to a substrate is achieved by binding interactions between the target molecule and substrate that are essentially distinct in character. Herein these binding interactions are attributable to first and second surface binding components that are present on the substrate surface.

The first surface binding component is some structural feature that has specific binding affinity with respect to the target molecule of interest. This means that the first surface binding component is capable of some form of binding interaction that is specific to the target molecule (or rather some region of the target molecule) as opposed to non-target molecule(s) that will be present in an analyte in admixture with the target molecule. Despite being suitably specific to the target molecule, the strength and specificity of this binding interaction is not useful for practical purposes since it is not effective in immobilizing the target molecule on the substrate under the prevailing conditions at which the substrate and analyte are exposed to one another. This is due to the nature of the binding interaction rather than any concentration effect associated with provision of the first surface binding component on the substrate surface. The nature of the interaction between the first surface binding component and target molecule may be dependent on the three dimensional structure (conformation) of the first surface binding component.

In contrast to the first surface binding component, the second surface binding component exhibits some non-specific binding to the target molecule. This means that the second binding component is capable of binding interactions based on one or more structural features that are common to the target molecule and to one or more, and possibly all, non-target molecules that are present in the analyte. The strength of this binding interaction is not effective to support stable binding of any particular molecule to the substrate surface. Examples of non-specific binding interactions include H-bonding, hydrophobic interactions, charge-charge interactions, dative (coordinate) bonds (for example involving metal-coordination complexes), and the like, and other potentially reversible reactions between reactive functionalities such as amine with carbonyl groups (imines), and carbohydrates with boronic acids.

It will be appreciated from this that the first surface binding component has the binding specificity to isolate a target molecule of interest but not the binding strength to do so (to be useful in a practical application), whereas the second surface binding component has neither the specificity nor the binding strength to isolate the target molecule. However, together the first and second surface binding components co-operate to provide an operative binding system that allows a target molecule to be successfully extracted from an analyte and that is effective for practical purposes. By suitable selection of first and second surface binding components in accordance with the present invention the binding efficacy of a substrate can be tailored with respect to a target molecules (or target molecules) that it is desired to isolate. Thus, the present invention provides a potentially powerful molecular-separation tool. The invention may have particular utility in the field of bio-separations.

For the avoidance of doubt, the binding interactions described are the binding interactions that will be prevalent at the prevailing conditions under which the substrate and analyte are exposed to each other. Invariably, the analyte will be a solution or suspension, possibly complex with respect to the species it contains.

In general terms, it is believed to be important that surface binding components are dimensionally smaller than the target molecule with which they can interact. This means that for the binding interaction between a surface binding component and target molecule to take place, the target molecule and substrate (upon which the surface binding component is present) must be in close proximity to each other. In turn, this provides the opportunity for other surface binding components present on the substrate surface to interact with the target molecule to effect selective and stable binding.

The surface binding component may be biological or non-biological in nature. In this context the term non-biological is intended to denote a species that retains functionality in the context of the present invention after storage under non-aqueous conditions and that is not denatured after heating, for example at 50° C. Examples of biological surface binding components include proteins, and longer chain peptides and oligonucleotides, and the like. Examples of non-biological surface binding components include functional groups or short polymer chains (usually organic in character), small molecule organic compounds, small molecule inorganic compounds, including metallic and organometallic complexes. The first and second surface binding components may both be biological or non-biological in character, or one component may be biological and the other non-biological in character. In certain applications, the use of an entirely non-biological (abiotic) surface capture agent may be preferred. Usually, the second surface binding component is non-biological in character.

As noted, the first and second surface binding components are necessarily different from each other with respect to the nature of the binding interaction possible with the target molecule. The first and second binding components may fall in the same general class, for example they may both be organometallic species, but the components are nevertheless not identical.

It is possible for the invention to rely on the use of more than two surface binding components to achieve stable binding of a target molecule, and the same principles will apply as described herein in relation to the selection and requisite function of the totality of surface binding components in this case. Thus, the invention embraces the use of a plurality of different surface binding components having the binding characteristics as per the first surface binding component, as described herein, and/or the use of a plurality of (different) surface binding components having the characteristics of the second surface binding component, as described herein. For ease of discussion, however, the present invention will be described with reference only to first and second surface binding components.

Herein, unless otherwise stated, term “target molecule” refers to a molecule that it is desired to immobilize on the substrate. The present invention has particular utility in the provision of surface capture agents and the target molecule may be any type of molecule that it is desired to extract from an analyte. The method of the present invention may be applied to immobilize a general class of target molecule from an analyte containing a variety of classes of non-target molecules, or to immobilize a specific type of target molecule from an analyte containing a mixture of non-target molecules falling within the same general class. The invention may in fact be tailored to provide suitably high binding specificity to immobilize a specific target molecule in admixture with other non-target molecules. Alternatively, the invention can also be applied to increase the relative population in an analyte of target molecule(s) with respect to non-target molecules.

The target molecule may be biological in character, such as proteins (e.g. an antibody) or, polypeptides or polynucleotides. By way of illustration, the present invention may be applied in practice to immobilize antibodies on a substrate.

In an embodiment of the invention, the target molecule is a pathogen and in the context of the present invention this term is used to embrace biological and non-biological agents that are capable of causing disease, and possibly death, in humans and/or other animals. The pathogen may be biological such as a virus or bacterium, and here the present invention may find utility in medical or veterinary applications. The pathogen may non-biological in character, such as a synthetic toxin or nerve agent, and here the present invention may find utility as a military tool or in the fight against terrorism.

Herein, unless otherwise stated, the term “substrate” is intended to mean a solid phase material that is a suitable platform for immobilizing the target molecule of interest. Generally the substrate used will be a synthetic substrate of a format commonly used in preexisting solid phase applications. The substrate may take any form. In biological applications the substrate will usually be in the form of beads, membranes, multi-well plates, slides, capillary columns or any other format that is used for biological assays, affinity separations, diagnostics or other applications where biological molecules are immobilized on some insoluble material (solid support). As will be described, depending upon the surface characteristics of the substrate, some modification of the substrate may be required in order to achieve efficacy or optimization of the method described herein.

In accordance with an embodiment of the present invention, the interaction between at least one of the first and second surface binding components and the target molecule is thermodynamically reversible so that binding of a target molecule to a substrate (by the combined effect both surface binding components) may be manipulated as might usefully be required. For example, when binding of the target molecule is contingent for stability purposes upon the binding interaction between a particular surface binding component and the target molecule and this interaction is pH dependent, variation in the pH may provide a convenient means of releasing the target molecule once captured on the substrate.

It may be preferred that the various surface binding components used in practice of the present invention are non-biological since this enables the provision of abiotic receptors or synthetic antibody mimics with far greater physical and chemical stability than those based on specific interaction-based approaches using proteins and oligonucleotides. This aspect of the present invention may have particular applicability in relation to developing temperature stable affinity columns for purification, synthetic capture agents used in protein microarrays and other diagnostic screening tools. It will be appreciated however that the underlying concepts of the invention are applicable to other applications that otherwise require high affinity/binding ligands on a substrate surface. Here it will be appreciated that this aspect of the present invention is quite distinct from certain prior art techniques that rely on solid supports that are supposed to have minimal non-specific interactions with a target molecule, and that do not rely on such interactions to influence the binding efficiency of a ligand or ligand combination that is provided on the support surface and that has specificity with respect to binding of the target molecule.

It will be appreciated from the foregoing that the present invention may be applied to provide high affinity and high specificity surface capture agents as a result of the combined binding effect of surface binding components that are individually unsuitable to achieve stable binding of a target molecule. In this context the term “surface capture agents” implies a combination of surface binding components that are provided on the surface of a substrate and that are capable, in combination, of effecting selective and stable binding and thus immobilization of a target molecule from a mixture of molecules in an analyte. This should be contrasted with conventional “capture agents” that rely on high binding affinity of a particular molecule or ligand to a particular target molecule in preference to other molecules within a mixture.

It will be appreciated that a surface capture agent in accordance with the present invention does not have a clearly defined high binding affinity structure/component like a conventional capture agent involving, for example, a peptide, oligonucleotide, or protein such as an antibody. In accordance with the present invention, binding efficacy correlates with the sum of all operating binding interactions due to first and second surface binding components present on the substrate surface, and is dependent on induced fit of these surface binding components with the target molecule.

In accordance with the present invention, suitably selective and stable binding may be based on the equilibrium state of reversible interactions involving a number of different types of surface binding components and a target molecule. With respect to stability, the preferred surface interactions will be dictated by thermodynamic/energetic considerations. Interaction of a particular surface binding component may be dominant over secondary interaction(s) involving other surface binding components and the target molecule, and in this case a selective and stable binding event will typically be contingent upon energetic considerations involving these secondary interactions under the prevailing conditions. In the event that the prevailing conditions do not favour these secondary interactions, they will not be available to contribute to binding of the target molecule. In this case, and in the absence of other suitable binding interactions involving other surface binding components that might be present, effective and stable binding of the target molecule will not take place.

Thus, in accordance with the present invention, the combination of surface binding components necessary to effect stable binding of a target molecule constitutes a dynamic combinatorial library of surfaces. With the approach of the present invention, the various components of a bio-mimetic surface can be designed to separately improve the association and dissociation rates associated with a binding event, as opposed to globally improving the dissociation constant (K_{D}=k₋₁/k₊₁).

Typically, an analyte from which it is desired to extract a target molecule will also contain buffers and/or preservatives, usually to stabilize the target molecule. For the invention to work as intended it is important that any buffer or preservative, or rather ligands/ions from the buffer or preservative, does not detrimentally interfere with binding interactions necessary to immobilize the target molecule on the substrate. For any given system it may be necessary to manipulate the surface binding components in order to ensure that the desired interactions prevail over interactions that would otherwise compromise the required binding interactions.

In practice, identification of surface binding components suitable for use in the present invention may be undertaken through a process of discovery using a library of different combinations of species. In accordance with this process the ability of a particular combination of surface binding components to facilitate binding of a particular target molecule to a particular substrate is assessed over a variety of different permutations based on the surface binding components used, the substrate, the target molecule and the prevailing conditions. In other words, the affinity of a target molecule to a substrate by interaction of both of these species through a combination of surface binding components may be assessed in order to identify combinations of variables that yield desirable results. By proceeding in this way it is in fact possible to rank combinations of variables according to observed binding efficacy to a given target molecule. This discovery process affords great flexibility in approach and forms part of the present invention. For example, it may be desired to produce an operative binding system based on a specific substrate. Here, in the discovery process the substrate is maintained constant throughout with other possible variants being manipulated in order to identify potentially useful combinations specific to that substrate. Variations of this general approach may be used in the discovery process to identify potentially useful systems specific to a given target molecule or to a desired combination of substrate and target molecule. It will be appreciated that this approach has extensive potential and scope without departing from the general concept underlying the invention, i.e. the use of a combination of surface binding components to achieve immobilization of a target molecule on a substrate.

This aspect of the present invention may be applied to provide high throughput processes to identify surface binding components that in combination may be used to generate high affinity and high specificity “surface capture agents”. Consistent with an embodiment described above, this aspect of the invention may be applied to modulate the overall binding efficacy with respect to a target molecule of a particular type of surface binding component (such as a discrete molecule or ligand) that is specific to a target molecule but unable to achieve stable binding of the target molecule via other non-specific binding interactions attributable to other surface binding components present at a substrate surface. While it is possible to reduce the surface down to a colloidal particle or a synthetic polymer, the phenomenon is essentially a surface interaction and it is not necessary for any component of the surface capture agent to be biologically relevant in solution.

The binding properties of large molecules such as polypeptides and proteins are non-monotonic (ie, require hydrophobic, hydrogen bonding, electrostatic interactions) and hence, are best met by complementary spatially distributed functionalities on the surface of a substrate. Identification of the correct functionalities, their number, ratio and spatial distribution is challenging as there are a huge number of options. The use of combinatorial libraries such as those described in PCT/AU03/00566 (published as WO 03/095494), it is possible to identify non-monotonic surfaces with some level of specificity for one protein in preference to another. By combining a surface binding component, such as a ligand, with one or more other surface binding components each having some relative specificity for a particular protein in preference to another, a high affinity surface capture agent can be identified for a particular target molecule. In this case, high affinity for some target molecule is a population phenomenon arising from combinations of weak binding interactions to a particular target molecule.

The prior art describes numerous procedures from the beginnings of combinatorial chemistry, where some minimal binding ligand having some measurable affinity and specificity, are extended through the use of monomers such as amino acids and spacers/linkers. The focus of such work has been the development of high affinity ligands that are effective in solution. Any secondary surface contributions are not considered an issue unless they lead to increased background noise or damage the binding capacity of the capture agent. In the alternative, prior art (US2004/0161798A1 by Thomas Kodadek) describes combining two ligands having distinct binding specificity for a target molecule on long (polyethylene glycol) spacers. Secondary surface contributions are not considered and the performance of the substrate is simply determined by spacer length and ligand density. Opposed to combining specific ligands to a target molecule, synthetic surfaces can also effectively act as a tertiary component of any interactions performed on that surface and they have the potential to damage or even destroy high affinity binding of surface immobilized capture agents. Recognizing the importance of surfaces in such applications, the present invention provides method for the generation of libraries of surface capture agents for a given target molecule, capable of binding to some specific binding site of a target molecule, which method comprises identifying some surface binding component (having some measurable affinity and specificity) to some portion of the binding site on a target molecule; the surface binding component being dimensionally smaller than the target molecule. Once identified, the surface binding component is generated on libraries of non-monotonic surface binding elements, and/or metal complexes and/or other potentially reversible interactions. By testing different libraries of surface binding elements in combination with some surface bound ligand, affinity and specificity to a target molecule can be progressively improved. Combining the surface binding component with libraries having combinations of one or more surface binding components and testing such combinations can identify surface capture agents having high total/collective affinity and specificity to a target molecule.

In accordance with the invention the binding effect with respect to a particular type of target molecule of the first surface binding component, as described herein, present on a substrate surface may be potentiated by inclusion on the substrate of another type of surface binding component. Through such modulation, poor affinity surfaces can be transformed into improved affinity surface capture agents that may be tuned in a pre-determined manner to give preferential increase in capture of the target molecule or that are designed to capture and release target molecules under specific conditions.

In accordance with the present invention a metal complex may be one example of a surface binding component. Herein metal complexes are metal species formed when a metal ion in solution forms coordinate covalent bonds (also called dative covalent bonds) with electron donor ligands also present in solution. Such ligands will be called herein coordination ligands, metal ligands or simply, ligands. The nature of the complex formed for any given metal will depend upon the ligands in solution as well as the ability of the ligands to form suitably stable associations with the metal ion. The ligands may be mono-, bi- or poly-dentate depending upon their structure and ability to interact with the metal ion thereby forming a complex. Hydrates and/or anions (also called counter ions) will invariably be part of the structure of the metal complex in solution.

In this case the mechanism by which the metal complex facilitates binding of a target molecule, or rather a region of the target molecule, is believed to involve displacement by the target molecule of one or more coordination ligands associated with the metal complex. For this to occur the target molecule must be able to form preferential associations with the metal ion of the metal complex when compared to one or more existing coordination ligands that are already present in association with the metal ion prior to interaction with the target molecule. It is possible in accordance with an embodiment of the invention to manipulate the binding characteristics of the metal ion with respect to the target molecule in order to achieve the desired binding interaction. Thus, in an embodiment of the invention one or more coordination ligands associated with the metal ion are selected in order to control binding of the target molecule as required.

The metal complex may facilitate binding to the substrate by a similar ligand displacement mechanism as described above in connection with the target molecule, and the binding characteristics of the metal ion with respect to the substrate may also be manipulated as necessary.

Given the mechanism proposed, it will be appreciated that the species formed when a metal ion binds a target molecule could be regarded as being a metal complex since when bound the target molecule is a coordination ligand associated with the metal ion. The same could be said for the species formed when a metal ion binds to a substrate. However, to avoid confusion, the term “metal complex” will be used herein to refer to a metal ion and associated coordinate ligands before any such binding events have taken place.

Herein, in this context and unless otherwise stated, the terms coordinate and bind, and coordination and binding interaction, are used interchangeably. Depending on the complex structure and the conditions of use, the strength of the coordinate bonds are tunable to support other surface binding components.

In its simplest form the metal complex comprises a central ion surrounded by a number of coordination ligands. However, the structure of the metal complex may be more complicated, involving two or more metal ions and a variety of associated coordination ligands. The metal ions may also form dative bonds with hydroxyl and hydronium ions and water. The structure of the complex and the nature of associated ligands is likely to influence the efficacy of the metal complex in achieving the necessary binding interactions, i.e the necessary coordination with the target molecule and the substrate, in order to achieve or contribute to immobilization of a target molecule on a substrate. As noted, it is believed that the active metal complex is capable of ligating to the substrate and the target molecule through a process of ligand exchange.

A range of metal complexes may be useful in practice of the present invention and variation of the metal complex represents a point of diversity that allows flexibility of practice of the present invention. As will be apparent, the underlying principles of the present invention affords a number of other points of diversity associated and this may enable enhanced selectivity in terms of immobilizing a target molecule.

It will be appreciated from the foregoing that the metal complex useful in practice of the present invention is one that is capable of undergoing ligand displacement thereby forming a binding interaction (i.e. coordinate bond) with the substrate and with the target molecule under the conditions (such as pH, temperature, ionic strength, etc.) at which these species are exposed to each other and under the conditions associated with an assay or other solid phase applications in which the methodology of the invention is employed.

It has been found that certain metal compounds result in complexes (in aqueous solution) that are generally useful as leads in the discovery process described above. Examples of metals that may be used include transition metals such as scandium, titanium, vanadium, chromium, ruthenium, manganese, palladium, iron, cobalt, nickel, copper, molybdenum and zinc. The salt moiety may be the chloride, acetate, bromide, nitrate, perchlorate, alum or sulphate, and it may be necessary to manipulate the salt moiety (anion) to identify the optimum binding conditions for a target molecule.

The effect of variables may of course be explored using the discovery process described herein to identify operative combinations that facilitate the immobilization of target molecules on a substrate using the surface binding components to provide suitable binding interaction between the target molecule and the substrate under specified (prevailing) conditions.

In a further embodiment of the invention the nature of the various coordination ligands making up the metal complex and “available” for displacement by a target molecule may also be controlled in order to manipulate overall binding strength under specified conditions, as required. For example, the process of discovery using a library of different combinations of species in the complex generates a diversity of metal complexes having different binding affinities including many that do not bind at all. Even in the case where one metal such as cobalt is chosen, a gradation of binding affinities to a given target molecule can be achieved by varying the type of coordination ligands present in the metal complex as well as other variables such as pH, ionic strength, etc. Thus, it is possible to modify (enhance or weaken) the binding affinity of the metal complex with respect to a particular target molecule and substrate. In this way it may be possible to identify metal complexes that can provide a suitable binding interaction with the substrate and with the target molecule under specified conditions but under slightly modified conditions, the binding interaction is unstable. As already described, this embodiment of the invention may be applied to achieve a stable binding interaction under some specified conditions to give some minimal threshold binding affinity.

In accordance with the present invention the substrate used may inherently have appropriate surface functionality that provides the surface binding components required, or provide binding sites to which one or more types of surface binding components may be attached. However, in an embodiment of the invention the substrate may be modified by the provision thereon of a coating that facilitates the necessary binding events associated with the present invention. The coating may includes suitable surface binding components or provide binding sites to which one or more types of surface binding components may be attached. The embodiment of the invention may also be used to increase the range of substrates that may be employed by rendering useful otherwise unsuitable substrates.

In a preferred aspect of this particular embodiment the coating takes the form of a polymer incorporating repeat units that include suitable functionality to provide one or more surface binding components or binding sites that will allow attachment of one or more surface binding components. The characteristics of the repeat unit may be derived from the monomers from which the polymer is formed, although the polymer may be formed and then modified to include pendant functional groups which impart desirable binding properties. The functional groups may be components of different repeat units in the polymeric chain but it is also possible that the functional groups are present within a single repeat unit. The polymer may also include other structural components in order to impart different functionality. This is discussed in more detail below.

One type of polymer that may be useful is a copolymer of first and second monomers as described in Applicant's published International patent application WO 03/095494 which is incorporated herein by reference.

In addition to the nature of the polymer being responsible for controlling binding of the metal complex, additive ligands may also be included to vary binding events by passive or dynamic interaction with the metal centre of the complex. Although illustrated with reference to the use of coated substrates the usefulness of such additive ligands is not limited to this particular embodiment of the invention. The additive ligands include but are not limited to electron rich donors such as amine containing molecules. Examples of such include ethylenediamine, tetramethyl ethylenediamine, iminodiacetic acid and oxalic acid which are multi-dentate ligands that form two or more dative covalent bonds with metal ions. Depending on the metal and initial coordination ligands, different additive ligands are preferred. It is believed that the resulting active metal complex formed, which may also contain dative bonds with hydroxyl and hydronium ions and water, is then capable of ligating to the polymer surface and target molecule through a process of ligand exchange.

The coating to be used in any given application may be selected from a library of coatings that has been generated based on the binding performance of the coating with respect to a target molecule or to a surface binding component to be provided on the coating (and on the binding performance of that surface binding component when so-provided). In accordance with this aspect of the present invention the coating such as discussed in WO 03/095494 may be used as a privileged scaffold for combinational library generation of polymer coatings.

Alternatively, there may be a structural template that acts as a base (structural platform) to which are attached the first and second binding components and, possibly, a linker. Herein this combination is described as a binding molecule. The role of the binding components has already been explained and the role of the linker is to facilitate binding of the binding molecule to the target substrate. Typically, this is done via a functional group in the linker that is reactive towards some structural feature, such as a functional group, present on the target substrate.

The structural template is usually designed or selected based on its ability to couple to the, first binding component and reversibly bind (or chelate) to the second binding component. It may be necessary for the first and second binding components to have a particular location on the structural template in order to be accessible for binding with a target molecule. It may also be necessary for the first and second binding components to have a particular spatial distribution relative to each other in order to facilitate the required binding event with a target molecule. The choice of structural template should take such considerations into account.

As foreshadowed above, the structural template may be, or include as an integral part thereof, one of the first and second binding components. Thus, the structural template may be a small molecule compound that exhibits some binding affinity and, possibly, selectivity for a target molecule. This binding affinity will be as per the characteristics of the first or second binding components as already discussed. In one case, the structural template may be, or include as an integral part thereof, a first binding component and have requisite functionality to bind a second binding component in a manner such that the ability of the first and second binding components to bind a target molecule is retained. For example, the structural template/first binding component can be a heterocyclic compound with some affinity and, possibly, selectivity for a target molecule. The second binding component may then be a metal complex that is able to chelate to the heterocyclic compound. Alternatively, the metal complex can chelate to a metal chelating ligand that is coupled to the heterocyclic compound. In either case, once chelated to the heterocyclic compound the resultant metal complex moiety retains the ability to interact (as per a second binding component) with the target molecule of interest.

One specific example of a heterocyclic compound that may be useful in this embodiment is triazines. A substituted triazine is a well known structure from which a huge diversity of different triazines can be synthesized. As previously discussed, identifying high affinity and high selectivity triazine analogs is a difficult process but identifying compounds with weak or medium affinity to a target molecule is far easier. One such medium affinity triazine that binds to the Protein A binding region on the Fc of an antibody is 2-(2-aminoethylamino)-4-anilino-6-tyramino-s-triazine (APA). (R Li, V. Dowd, D. J. Stewart, S. J. Burton & C. R. Lowe, Nature Biotechnology 16, 1998, 190-193; Machius, M., Vertesy, L., Huber, R., and Wiegand, G. (1996) Nature. Biotechnology. 16, 190-195). This triazine template also chelates a metal complex (the second binding component) and the resultant binding molecule has been found to exhibit far greater affinity and selectively to an antibody than either binding components acting alone. Similarly, triazines with even weaker binding affinity and selectivity could be converted to high affinity and high selectivity ligands (binding molecules) for the Fc of an antibody (see FIG. 1). Similarly, in accordance with the present invention any small molecule template having some affinity and, possibly selectivity, to any target molecule can be significantly improved by addition of a suitable second binding component.

In another embodiment, the structural template can be used as a structural template to which individual first and second binding components can be bound. It is fundamental of course that once bound to the structural template the binding components retain their intended binding functionality with respect to the target molecule of interest. In one example, the structural template can be a chelating agent that binds a metal complex (as a second binding component) as well as having the functionalities to couple a small molecule ligand (as a first binding component). There are a number of chelating agents that can act as a structural template in this regard, such as nitrilotriacetic acid, poly Histidine tags, or ring systems such as 1-acetato-4-benzyl-triazacyclononane and its analogs (D. L. Johnson and L. L. Martin, (2005) J. Am. Chem. Soc., 127, 2018-2019). Like triazines, such structural templates allow a huge diversity of different analogs to be synthesized and used as binding molecules having first and second binding components.

As foreshadowed and if required, a linker can be incorporated into the structural templates as described above and will typically include a functional group that is reactive towards some structural feature of the target substrate. Invariably, the linker is not an intrinsic part of the structural template. Thus, linker is typically bound to the structural template by reaction with some structural feature of the structural template.

In the present invention, a surface binding component of a specific binding nature needs to be included, such as one that has been designed in accordance with the pharmacophore approach described in Applicants own co-pending International patent application no. PCT/AU2004/001747 (published as WO 2005/057462) the content of which is incorporated herein by reference and described below. The pharmacophore approach is particularly useful where the target molecule is a protein since it enables binding to a specific region thereof. Once identified the small molecule may be provided on the substrate surface, possibly via the kind of coating approach described above.

The present invention also extends to substrates that comprise first and second surface binding components and that are suitable for use in the method of the present invention. The invention embraces substrates that have also been modified or manipulated in accordance with embodiments described herein by inclusion of a suitable surface coating and/or structural features to enable selectivity to the target molecule. The present invention also extends to the use of such substrates, possibly in combination with other embodiments as described, in an assay to immobilize a target molecule. Yet further, the invention embraces the species formed when a target molecule becomes bound to a substrate in accordance with the method of the invention as described herein.

In another embodiment of the present invention there is provided a method of designing a capture agent, which comprises identifying first and second surface binding components that are capable of achieving a desired binding interaction between a substrate and a target molecule. The invention also relates to the practical application of such capture agents.

As noted above, the substrate may include further surface binding components that have the same binding effect as the first and/or second surface binding components, as described herein. The various embodiments of the present invention should be understood with this point in mind.

### BRIEF DISCUSSION OF FIGURES

Embodiments of the present invention are illustrated in the accompanying drawings in which:

FIG. 1 illustrates examples of first binding components (six triazine analogs) that may be useful in implementation of the present invention for the case of antibodies;

FIG. 2 is a bar chart illustrating Net Relative Fluorescense Intensities (RFI) that result from IgG binding with APA-4 analogs, metal complexes and their combinations on a DMA-NAS polymer coating as obtained in Example 1 included herein;

FIGS. 3, 4 and 5 are bar charts illustrating Net Relative Fluorescence Intensities (RFI) that result from the binding competition on various surface compositions of Human IgG with HSA, HDL and transferrin as obtained in Examples 2 included herein;

FIG. 6 is a bar chart illustrating the binding intensities of Cy3 IgG (shown as positive or above zero) and Cy5 HSA (shown as negative or below zero) on the various Apa analogs, metal complexes and their combinations. Each intensity column is the average of six replicates, the positive values being the intensity resulting from the IgG binding while the negative values are the intensity from HSA binding as obtained in Example 3 included herein;

FIG. 7 illustrates binding of IgG to APA or the metal complex alone is poor or non-existent. However, in combination, some APA/Co combinations give far higher binding. Various combinations of different metals and small molecule ligands give synergistic binding as obtained in Example 4 included herein;

FIG. 8 illustrates synergistic binding arising from the invention giving superior assay sensitivity compared to conventional amide coupling methods as obtained in Example 5, included herein.

Embodiments of the present invention are illustrated with reference to the following non-limiting examples.

### EXAMPLE 1

### Binding on Glass Microarrays

Metal Complexes

A 0.5M stock solution of each metal complex in DMF was prepared by adding the required amount in grams of metal salt as given below into 10 ml of DMF. All reagents were purchased from Sigma-Aldrich.
Name/Weight (g)./.(Catalog No)./.CAS
Cobalt(II) perchlorate hexahydrate/1.83/(401404)/13478-33-6
Copper(II) bromide/1.12/(437867)/7789-45-9
Iron(III) chloride hexahydrate/1.35/(236489)/10025-77-1
Nickel(II) perchlorate hexahydrate/1.83/(309338)/13520-61-1
Palladium(II) bromide/1.33/(205877)/13444-94-5

Ligands (APA Analogs)

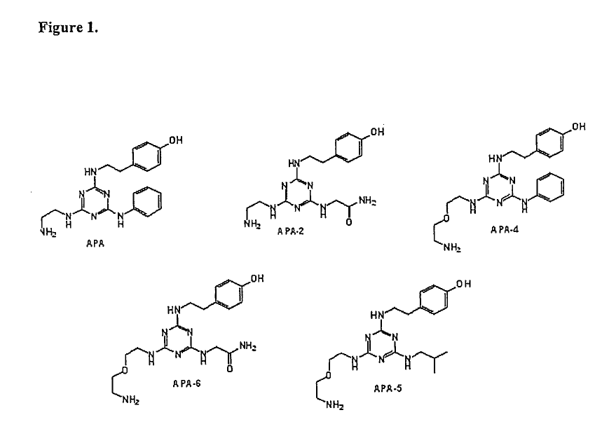

2-(2-aminoethylamino)-4-anilino-6-tyramino-s-triazine (APA), a Protein A mimetic that weakly binds to the Fc of an antibody (R Li, V. Dowd, D. J. Stewart, S. J. Burton & C. R. Lowe, Nature Biotechnology 16, 1998, 190-193 was used as an example of a first binding component. The synthesis was performed as described by the authors. A set of analogs as depicted in FIG. 1 were also synthesized. The use of APA-4 is reported in this example.

Polymer Coating (DMA-NAS Copolymer)

A copolymer of dimethylacrylamide (DMA) and N-acryloyloxysuccinimide (NAS) in ratio of 1:2 is used as an example of a polymer coating. The active ester of NAS is able to react with aminosilanized slides as well as with any amine containing ligands (first binding component), as described below.

Glass slides were washed 3 times with high purity methanol for 15 minutes with sonication and washed 2 times with HPLC grade Propan-2-ol for 15 minutes with sonication. The slides were rinsed 4 times with Sartorius water before being soaked in Aqua Regia solution for 30 minutes. The Aqua Regia mixture was prepared by adding 125 mL of 30% Nitric acid to 375 mL of 30% Hydrochloric acid. The Aqua Regia wash was then followed by 4 rinses with Sartorius water. Slides were dried under nitrogen flow.

Aminosilanization of Glass Slide.

The clean slides were then placed into 1% 3-aminopropyltrimethoxysilane (Sigma Aldrich Cat: 281778, 97%) solution in 95% acetone/water and shaken on a RATEK platform mixer for 30 minutes. The slides were then washed 4×5 minutes in 95% acetone/water, dried at 110° C. for 45 minutes in an oven under nitrogen and left to cool for 1 hour.

Dip Coating Slides with DMA-NAS

A 1% w/v solution of DMA-NAS in DMF was prepared by adding 2 g of DMA-NAS into 200 mL of DMF. The solution was mixed until dissolved. The slides were left to stand in the 1% w/v DMA-NAS solution for 3 hours with shaking. Then, the slides were washed 2 times in DMF for 2 minutes with shaking followed by a Propan-2-ol wash for 2 minutes with shaking. After being dried under nitrogen flow, the slides were used immediately for reacting spotted triazine amines.

Microarray Printing onto Amine Reactive Polymer Coated Slides

Preparation of Spotted Solutions

Metal complexes and ligands were made up to a final concentration of 10 mM in DMF and spotted onto the slides either individually or as mixtures. For the latter, 20 mM solutions of both metal complexes and ligands are first freshly prepared. To make the metal complex solutions at 20 mM, 40 uL of the 0.5M metal complex stock solution is added to 960 uL of DMF in a 1.5 mL Eppendorf tube. To make the APA-4 solutions at 20 mM, 6.56 mg of APA-4 was weighed into 1.5 mL Eppendorf tubes and 800 uL of DMF was added. Solutions were sonicated until completely dissolved. The solutions were then mixed in a polypropylene plate to a final concentration of 10 mM. For solutions containing either metal complexes or ligands, 50 uL of the 20 mM solution was dispensed into the wells and 50 uL of DMF was added. For solutions containing mixture of metal complexes and ligands, 50 uL of each 20 mM solution making the mixture was dispensed into the wells.

Microarray Printing

The APA analogs with or without metal complexes were printed onto amine reactive coated slides using the Chip Writer Compact printing robot (Bio-Rad) with 100 um stealth micro spotting pins (ArrayIt, Cat: SMP3). The sonicator bath was filled with DMF, the wash bath filled with propanol, and the humidity control was set to 20%. Prior each dipping, 2 wash cycles were performed as follow: Sonicate (DMF) for 150 seconds, Wash (propanol) for 150 seconds, and Vacuum for 60 seconds. After printing, the slides were placed into a storage container and stored in a low humidity cabinet until required for the binding studies.

Binding Assays.

Protein Labelling

Human IgG (Sigma Aldrich I4506 IgG from human serum 85K7545) was labelled with Alexa Fluor 555 (spectrally similar to Cy3 dye) labelling kit from Invitrogen (Invitrogen, Cat A20174) according to the standard procedure from the manufacturer and stored at 4 C until used.

Protein Incubation

The spotted slides were washed in DMF and in Propan-2-ol for 5 minutes each with shaking on RATEK platform mixer. Then the slides were immersed in a 10 mM Ethanolamine solution in water for 90 minutes. The 10 mM Ethanolamine solution was prepared by adding and mixing 302 uL of Ethanolamine (Lancaster, Cat 205-483-3) into 500 mL of water (Sartorius ultrapure). The slides were spun dry using a 2 slide micro centrifuge.

Each individual slide was inserted into a ProPlate individual module (Molecular Probes, Cat P37001) as per manufacturer instructions, and placed into the slide trays as needed (Molecular Probes, Cat P37003). 200 uL of 10 mM PBS buffer pH7.4 was added to each well and trays were placed onto a GFL 3023 plate shaker (Progen Scientific) and shaken for 1 hour. Wells were emptied by pipetting and 50 ul of Human IgG solutions with concentrations of 50 ug/mL were added to individual wells respectively. After 1 hour of incubation protected from light and with shaking onto a GFL 3023 plate shaker (Progen Scientific), wells were emptied using a pipette. Then 200 uL of 10 mM PBS pH7.4 with 0.05% Tween 20 was added to individual wells and shaken for 5 minutes. The slides were removed from ProPlate individual module as per manufacturer instructions, and washed for 5 minutes in 10 mM PBS pH7.4 with 0.05% Tween 20. Finally, slides were spun dried and were ready for fluorescence scanning.

Fluorescence Scanning and Analysis

The arrayWoRx Biochip Reader (AppliedPrecision) was used to scan the slides in the CY3 and CY5 channels as required with an exposure of 0.1, the sensitivity set to “High SNR” and a resolution of 26. The softWoRx® Explorer 1.1 (AppliedPrecision) software was used to export the images in TIFF format with grey scale using the File Min and Max (0 to 65,535). The Array-Pro Analyser V4.5.1.48 (Media Cybernetics) was used to extract spot intensities. Background intensities were extracted for each individual spot by using a local ring with an offset a 3 pixels and a width of 5 pixels. The trimmed mean was used to calculate the intensity of individual spot and background and the mean of the repeated spots was used to quantify the Raw and Net intensity observed for a given surface composition.

### RESULTS FROM EXAMPLE 1

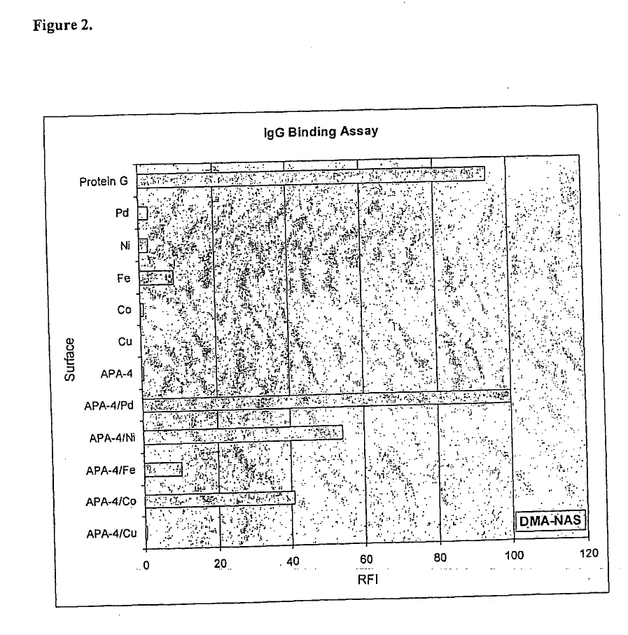

The graph in FIG. 2 shows the net Relative Fluorescence Intensities (RFI) that result from the Human IgG binding with the APA-4 analog, metal complexes and their combinations on the DMA-NAS polymer coating. Each intensity column is the average of six replicates. The column on the top corresponds to the binding with Protein G immobilized via amide coupling onto the surface. Protein G is known to be selective toward the Fc region of antibodies and is used as a reference to compare the binding of the synthetic surfaces. The fluorescence intensities resulting from the binding on the DMA-NAS polymer with Metal complexes or from DMA-NAS polymer with immobilized APA-4 are all relatively low, i.e. below 10 RFI. However, synergetic effects are observed when the DMA-NAS polymer, APA-4 and metal complexes are combined with the exception of APA-4/Cu. The surface resulting from the combination of APA-4, Palladium and DMA-NAS polymer shows a binding greater than that observed with Protein G. This example demonstrates the synergetic effects resulting from the combinations of the three components.

### EXAMPLE 2

### Competitive Binding on Glass Microarrays A

Polymers, coating methods, microarray printing, fluorescence scanning and analysis are as described in Example 1.

Metal Complexes

A 0.5M stock solution of each metal complex in DMF was prepared by adding the required amount in grams of metal salt as given below into 10 ml of DMF. All reagents were purchased from Sigma-Aldrich.
Name/Weight (g)./.(Catalog No)./.CAS
Cobalt(II) perchlorate hexahydrate/1.83/(401404)/13478-33-6
Iron(III) chloride hexahydrate/1.35/(236489)/10025-77-1
Nickel(II) perchlorate hexahydrate/1.83/(309338)/13520-61-1
Palladium(II) bromide/1.33/(205877)/13444-94-5

Ligands

APA Analogs:

See Example 1. APA-4 and APA-5 were used in this example.

Short Peptides

A 13 mer cyclic peptide that inhibits protein A binding with a Ki of 25 nM was identified by Weiss et al (Weiss, G A, Wells, J A and Sidhu, S S (2000) Protein Science 9, 647-654).

For this study, shorter sequences were synthesized and in particular the following two peptides are reported in this example:

FcP2F2: Ac-HIS-LEU-GLY-GLU-LEU-VAL-LYS-NH2

FcP2F1: Ac-GLU-LEU-VAL-LYS-NH2

Avidity Binding Assay

Protein Labelling and Solution Preparation

Human IgG (Sigma Aldrich, Cat I4506 IgG from human serum 85K7545) was labelled with Alexa Flour 555 (spectrally similar to Cy3 dye) labelling kit from Invitrogen (Invitrogen, Cat A20174) according to the standard procedure from the manufacturer and stored at 4 C until used.

Albumin Human Plasma (HSA) (Sigma Aldrich, A3782), High Density Lipoproteins (HDL) (Merck, 437641), and Transferrin (Sigma Aldrich, T8158) were individually labelled with Alexa Fluor 647 (spectrally similar to Cy5 dye) labelling kit from Invitrogen (Invitrogen, Cat A20173) according to the standard procedure from the manufacturer and stored at 4 C until used. Before performing the competition assay, equi molar solutions of protein mixtures were prepared in 10 mM PBS pH7.4 with 0.05% Tween 20 with a final concentration for each protein of 66 nM.

Protein Incubation

The spotted slides were washed in DMF and in Propan-2-ol for 5 minutes each with shaking. Then the slides were immersed in a 10 mM Ethanolamine solution in water for 90 minutes, washed and spun dried.

Each individual slide was inserted into a ProPlate individual module (Molecular Probes, Cat P37001) as per manufacturer instructions, and placed into the slide trays as needed (Molecular Probes, Cat P37003). 200 uL of 10 mM PBS buffer pH7.4 was added to each well and trays were placed onto a GFL 3023 plate shaker (Progen Scientific) and shaken for 1 hour. Wells were empty by pipetting and 40 ul of protein solutions were added to individual wells. After 1 hour of incubation protected from light and with shaking, wells were empty using a pipette. Then 200 uL of 10 mM PBS pH7.4 with 0.05% Tween 20 was added to individual wells and shaken for 5 minutes. The slides were removed from ProPlate individual module as per manufacturer instructions, and washed for 5 minutes in 10 mM PBS pH7.4 with 0.05% Tween 20. Finally, slides were spun dried and were ready for fluorescence scanning.

### RESULTS FROM EXAMPLE 2

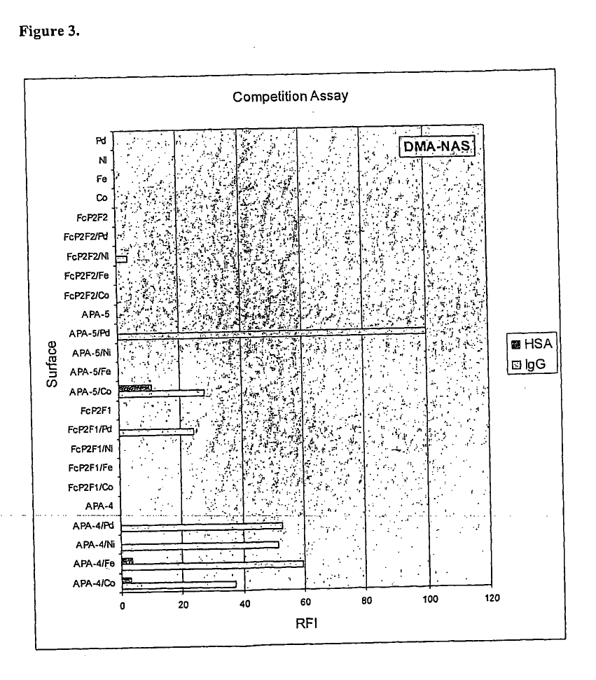

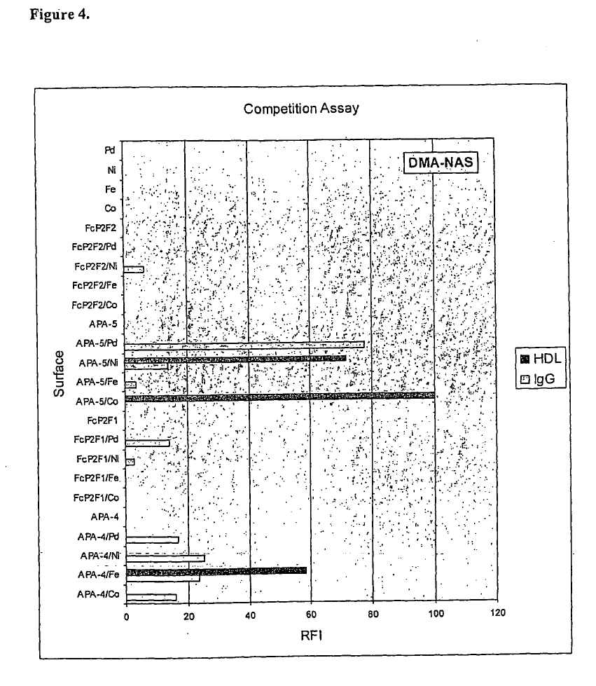

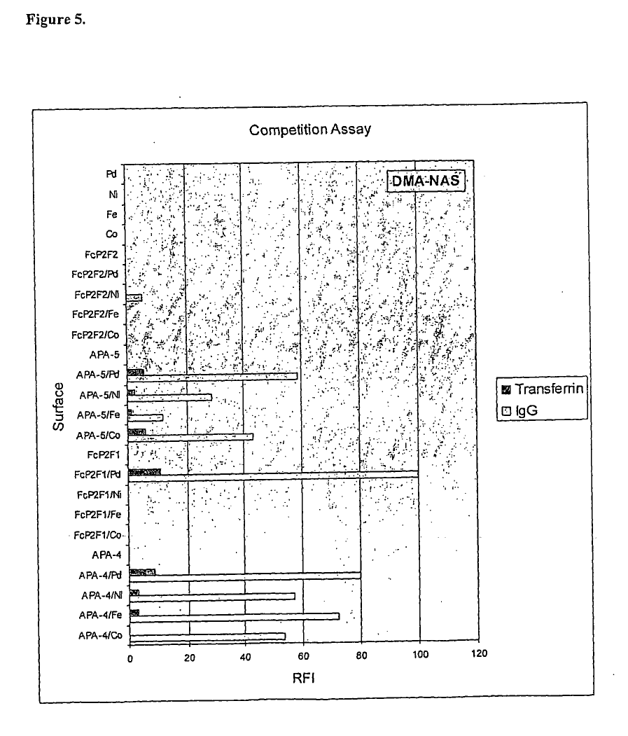

FIGS. 3, 4 and 5 show the net Relative Fluorescence Intensities (RFI) that result from the binding competition on various surface compositions of Human IgG with HSA, HDL and transferrin, respectively. HSA, HDL and transferrin are three abundant plasma proteins. Each intensity column is the average of five replicates. For each surface, the binding of the IgG and the plasma protein is reported. Table 1 gives the IgG selectivity of selected surface compositions combined with DMA-NAS polymer. The selectivity was defined as the relative amount of IgG binding compare to the total protein binding. Values under 50 mean that the surface is not selective for the IgG.

In the 3 assays, binding of the IgG or the plasma protein on the DMA-NAS polymer combined with the metal or the ligand were not detectable.

For the FcP2F2 peptide, only the combination with Nickel gave a very low selective binding for the IgG. For the FcP2F1 peptide, the combination with Palladium seems to be the best formulation with a good selectivity. The same is observed for the APA-5/Pd formulation. Other combinations such as with Nickel or Cobalt give partial selectivity. Finally, the APA-4/Co, APA-4/Ni and APA-4/Pd on DMA-NAS are good examples of muticomponent IgG selective surfaces.

TABLE 1
IgG selectivity of selected surface
compositions with DMA-NAS polymer.

| | Albumin (HSA) | HDL | Transferrin |
|---|---|---|---|
| APA-4/Co | 92 | 100 | 100 |
| APA-4/Fe | 94 | 29 | 96 |
| APA-4/Ni | 100 | 100 | 95 |
| APA-4/Pd | 100 | 100 | 90 |
| FcP2F1/Pd | 100 | 100 | 90 |
| APA-5/Co | 72 | 0 | 88 |
| APA-5/Fe | | 100 | 89 |
| APA-5/Ni | | 16 | 92 |
| ARA-5/Pd | 100 | 100 | 91 |
| FcP2F2/Ni | 100 | 100 | 100 |

This example demonstrates the binding synergetic effects resulting from the combinations of the three surface components and that selectivity can be obtained.

### EXAMPLE 3

### Competitive Binding on Glass Microarrays B

Similar coating methods, microarray printing, fluorescence scanning and analysis as described in Example 2 are used.

Polymer Coating (Activated Polyacrylic Acid)

A 2% solution of polyacrylic acid in DNF was activated using 1,3-diisopropylcarbodiimide (DIC) and N-hydroxysuccinimide (NHS). The slides were immersed in this activated solution for 30 mins and then excess activated solution removed by washing in DMF. Similar to Example 2, the slides were immediately loaded onto the printing robot and spotted with various concentrations of Apa analogs with or without metal complexes. The slides were washed 1×5 min DMF, 1×15 min isopropanol, 1×10 min water, 1×10 mins 100 mM bicarbonate buffer (pH8.9) and 1×60 min PBS/Tween (0.05%) to pre-swell to surface layer. Then the slides were placed into a storage container and stored at low humidity until required for the binding studies.

Avidity Binding Assay.

Human Serum Albumin (HSA, Merck Cat No: 12666, lot no: B58585) was labelled with Cy5 labelling kit from Amersham (GE biosciences Cat No: PA35000 Lot No: 332205) according to the standard procedure from the manufacturer and stored at 4 C until used. Similarly, Human IgG (Sigma Aldrich I2511 IgG from human serum 065K4888) was labelled with Cy3 labelling kit from Amersham (GE Biosciences Cat No: PA33000 Lot No: 332197).

The spotted slides were exposed to a mixture of labeled human antibody (IgG) with human serum albumin (HSA) in a ratio (of 1:25) characteristic to that in blood (30 nM and 750 nM, respectively). After 1 hr incubation, the slides were washed 2×5 mins with a Wash Buffer comprising 10 mM PBS pH7.4 containing 0.05% Tween20. The spot intensities (1 to 255) in Cy3 IgG (green) and Cy5 HSA (red) regions were extracted using an ArrayPro Analyser software (Media cybernetics V 4.5).

### RESULTS FROM EXAMPLE 3

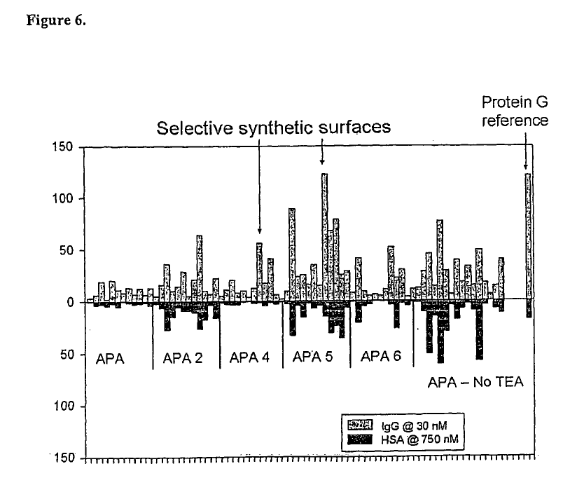

In FIG. 6, shows the binding intensities of Cy3 IgG (shown as positive or above zero) and Cy5 HSA (shown as negative or below zero) on the various Apa analogs, metal complexes and their combinations. Each intensity column is the average of six replicates, the positive values being the intensity resulting from the IgG binding while the negative values are the intensity from HSA binding. The column on the far right corresponds to the binding with Protein G immobilized via amide coupling onto the surface. Protein G is known to be selective toward the Fc region of antibodies and is used as a reference to compare the selectivity of the synthetic surfaces. A number of surfaces display good selectivity including one that is as good as protein G with similar loadings (intensities).

### EXAMPLE 4

### Surface Plasmon Resonance on Gold

Polymer Coating

Amino Functionalization with Cysteamine

Gold slides (GWC Technologies SPRchip Cat #SPR-1000-050) 18 mm×18 mm were amino-functionalized with Cysteamine (Fluka BioChemika Catalog #30070 MW77.15). 10 mL of 10 mM Cysteamine in Ethanol was prepared fresh and each slide placed in the 10 mL solution. Slides were incubated in Cysteamine overnight with constant gentle agitation.

Covalent Coupling of Polyacrylic Acid

Slides were then rinsed in Ethanol and incubated in a solution of 1% Polyacrylic acid. 2% w/v Polyacrylic acid was prepared by adding 6 mL of Stock 10% w/v PAA solution (Poly(Acrylic Acid) MW 50K 10% w/v solution in DMF) to 30 mL of dry DMF (N,N-Dimethylformamide (DMF) Labscan HPLC Grade—Dried over sieves and filtered). 100 mM solutions of NHS and DIC were prepared in dry DMF (N-Hydroxysuccinimide (NHS) (Aldrich cat: 130672), N,N′-Diisopropylcarbodiimide (DIC) (Sigma Aldrich cat: D125407)). 15 mL of 100 mM NHS and 15 mL of 100 mM DIC were added to 30 mL of 2% w/v of PAA solution and shaken on a RATEK platform mixer with speed setting 70 for 10 minutes. This gave a final 1.0% w/v solution of activated PAA. Each amino functionalized gold slide was placed in 10 mL of activated 1% wlv PAA solution and allowed to shake on a RATEK platform mixer with speed setting 70 C for 1 hour. The slides were then rinsed with dry DMF and using house nitrogen with a filter pipette tip in the end of the hose, the slides were blown dry using a concentrated flow over each slide. The slides were immediately loaded into the robot and printing commenced with APA analogues and Co. APA analogues and Co solutions were prepared according to Table 2. Upon completion of printing, slides were stored under nitrogen at 4 C.

TABLE 2
Apa analogs and Co complexes tested by SPR.

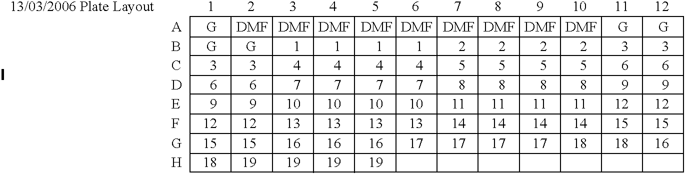

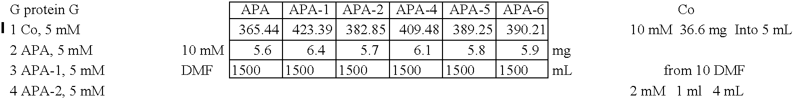

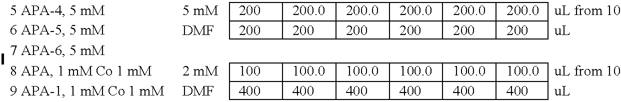

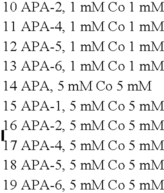

Gold Slide Pre-Swell

Prior to SPR analysis the printed slide was soaked in dry DMF for 10 minutes. All rinses were carried out in a glass petri-dish using the RATEK platform mixer with speed setting 50. Following the 10 minute DMF rinse the slide was soaked in iso-propanol (Lab-Scan Propan-2-ol, HPLC grade #C2519U) for 15 minutes. The iso-propanol wash was followed by a 10 minute water wash, a 10 minute 0.1M Sodium Bicarbonate pH8.0 wash, and a final 10 mM PBS soak for 1 hour. The GWC technologies SPRimagerII was allowed to warm up for an hour prior to use. The pre-swelled gold slide was taken directly out of the final PBS wash and mounted into the prism flow cell assembly (page 22, SPRimagerII User manual v2.1.50) and connected to the pump tubing. The flow cell was then filled with running buffer; 10 mM Phosphate Buffered Saline pH7.4, with 0.05% Tween20 and the angle of incident light on the prism was optimized to allow SPR to be performed on the coated gold SPR chip.

SPR IgG Binding Assay

Human IgG (Sigma #I2511, IgG from Human Serum >95% purity, 5.7 mg/mL) solutions were prepared in 10 mM Phosphate Buffered Saline pH7.4 with 0.05% Tween20. 1 mL volumes of 100 ug/mL, 50 ug/mL, 25 ug/mL and 10 ug/mL of IgG in PBS+0.05% Tween20 were prepared fresh and PBS+0.05% Tween20 was used as a “blank”.

The first concentration of IgG to be flowed through the cell and over the slide was 10 μg/mL. The approximate flow rate was 100 μL/min and the IgG solution was recirculated through the pump and flow cell. Images and data points were collected every 120 seconds for approximately 8000 seconds until the next increasing IgG concentration was flowed over the slide. After the final concentration of 100 μg/mL IgG was analyzed, the signal was monitored by flowing 10 mM Phosphate Buffered Saline pH7.4 with 0.2% Tween20 over the slide. The PBS+0.2% Tween was recirculated through the flow cell and data points were collected every 120 seconds for an overnight period.

### RESULTS FROM EXAMPLE 4

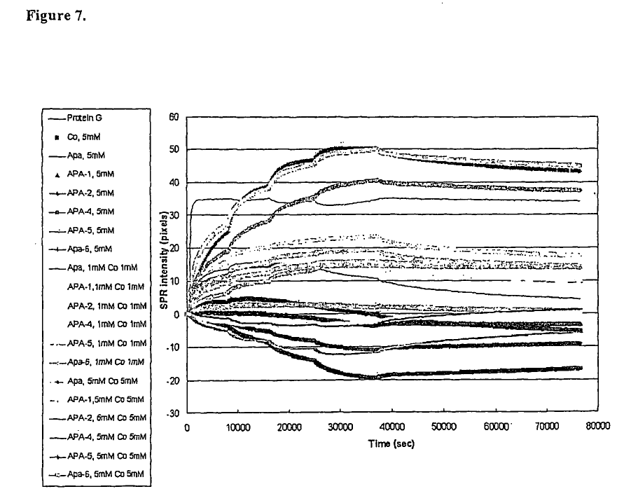

In FIG. 7 is shown the data obtained for the various spots of six different APA analogues and Co complex combinations. As shown in the antibody loading studies in previous examples, binding of APA or the metal complex alone is poor or non-existent. However, in combination, some APA/Co combinations give far higher binding. Various combinations of different metals and small molecule ligands give synergistic binding.

### EXAMPLE 5

### Sandwich Assay on Beads

Preparation of APA Cobalt Dynal Beads

Dynal M-270 COOH beads (200 μL, Dynabeads M-270 carboxylic acid beads Cat #143.05, 2×10⁹ beads/mL) were dispensed into a 1.5 mL microfuge tube. The beads were then washed with 3×200 μL water, followed by 3 washes with 200 μL of dry DMF (N,N-Dimethylformamide (DMF) Labscan HPLC Grade—Dried over sieves and filtered). The beads were then split into 6 separate 20 μL aliquots.

Stock solutions of 120 mM DIC and HOBt in DMF were then prepared. Add 9.3 μL of DIC (N,N′-Diisopropylcarbodiimide (DIC) (Sigma Aldrich cat: D125407) FWt 126.2, d 0.806) to 500 μL of dry DMF. Similarly, 8.1 mg of HOBt (AusPep 1-Hydroxybenzotriazole (HOBt) MW: 135.13 Cat No. 4005A) was added to 500 μL of dry DMF.

To each 20 μL bead pellet, 20 μL of 120 mM HOBt solution was added. The bead solution was then vortexed to form a suspension and then 20 μL of 120 mM DIC solution was added. The beads were allowed to stand for 15 minutes with occasional vortexing. Following the 15 minute incubation the beads were washed twice with 50 μL of dry DMF.

Preparation of Stock Solutions of Apa-Amine and Cobalt Complex

A 10 mM solution of Apa-NH₂ (2-(2-aminoethylamino)-4-anilino-6-tyramino-s-triazine (Apa), FWt 365.4) was prepared by adding 7.31 mg to 2 mL of dry DMF. 10 mM Cobalt perchlorate.hexahydrate (FWt 365.9) was prepared by adding 7.3 mg to 2 mL of dry DMF. 100 μL of each solution was combined to form a 5 mM stock solution of the Apa-NH2-Cobalt mixture.

From the 5 mM Apa-NH2-Cobalt stock solution, the following solutions were prepared:
2.5 mM, Add 100 μL of 5 mM Apa-NH₂-Cobalt to 100 μL of dry DMF;
0.5 mM, Add 20 μL of 5 mM Apa-NH₂-Cobalt to 180 μL of dry DMF;
0.25 mM, Add 10 μL of 5 mM Apa-NH₂-Cobalt to 190 μL of dry DMF;
0.01 mM, Add 4 μL of 5 mM Apa-NH₂-Cobalt to 196 μL of dry DMF;
0.05 mM, Add 2 μL of 5 mM Apa-NH₂-Cobalt to 198 μL of dry DMF; and
0.025 mM, Add 10 μL of 0.5 mM. Apa-NH₂-Cobalt to 190 μL of dry DMF.

50 μL of the resultant solutions were added to each bead pellet and vortex mixed. The reaction mixture was placed on a rotor overnight. The following day the bead solutions were washed 3 times with 100 μL of dry DMF then 3 times with 100 μL of water. The beads were resuspended in 100 μL of water.

Coating TNF-α Antibody onto APA-Co Dynal Beads

50 μL aliquots of the APA-Co beads were dispensed into separate centrifuge tubes. The beads were concentrated and the supernatant removed. A 400 μL solution of 50 μg/mL TNF-α capture antibody (BD Biosciences Cat #551225, Mouse TNF Capture Antibody, 0.5 mg/mL) was prepared by diluting 40 μL of TNF-α Antibody with 360 μL of 10 mM Acetate Buffered Saline pH4.0. 50 μL of 50 μg/mL TNF-α capture Antibody solution was added to each of the 6 tubes containing APA-Cobalt bead pellets. The beads were vortex mixed and sonicated. The beads were placed on a rotator for one hour. Following the one hour incubation, the beads were washed 3 times with 50 μL of saline. The beads were then stored in saline at 4 C.

TNF-α Antibody Loading on Dynal Beads

The TNF-α antibody loading assay on Dynal beads was performed according to the Luminex procedure. In brief, the materials and methods are as described.

Assay Components:
Antibody coupled beads: Add 10 μL of TNF coated APA-Co bead concentrate to 1000 μL of Assay Buffer
Goat anti-mouse IgG R-PE conjugate (Biosource Goat F(ab′)2 Anti-Mouse Ig's, R-PE conjugate 1 mg/mL, Cat #AMI4407) diluted to 0.5 μg/mL in Assay Buffer

Wash/Assay Buffer: 10 mM PBS pH7.4 containing 1% BSA and 0.05% Tween20

Assay Protocol.

Pre-wet the filter plate (Millipore Multiscreen HTS plate: #MABVN1250) by placing 100 μL of Assay Buffer into each well, incubate for 15 minutes then apply vacuum sufficient to gently empty the wells. Add 20 μL of TNF-α APA-Co beads to the appropriate microtitre wells. Add 100 μL of the diluted Goat anti-mouse IgG R-PE conjugate to the appropriate microtiter wells. Shake the filer plate at room temperature at 500 rpm for 1 hour in the dark. Remove the solution from all wells by applying vacuum sufficient to gently empty the wells. Add 100 μL of Wash Buffer into each well and apply vacuum sufficient to gently empty the wells. Repeat wash procedure, then add 100 μL of Wash Buffer to each well and shake for 60 seconds. Load the plate into the Luminex XYP™ platform and read.

The TNF-α Assay on Dynal beads was performed using a Chemiluminescent Sandwich Immunoassay. In brief, the materials and methods are as described.

Assay Components:
Antibody coupled beads: Add 10 μL of TNF coated APA-Co bead concentrate to 1000 μL of Assay Buffer
Detection Antibody: Detection anti-TNF monoclonal antibody (BDBiosciences cat #554415 Mouse TNF-biotin Detection Antibody, 1.0 mg/mL). Working solution was 0.5 μg/mL in 10 mM Phosphate Buffered Saline pH7.4 containing 1% w/v Bovine Serum Albumin (BSA Sigma Cat #A3059) and 0.05% Tween20TNF-α
Antigen standards were prepared in 10 mM PBS pH7.4 containing 1% BSA and 0.05% Tween20
Streptavidin-HRP (Sigma Cat #5512) working solution was 0.5 μg/mL in 10 mM PBS pH7.4 containing 1% BSA and 0.05% Tween20
Assay Buffer: 10 mM PBS pH7.4 containing 1 % BSA and 0.05% Tween20
Wash Buffer: 10 mM PBS pH7.4 containing 0.05% Tween20
Chemiluminescent substrate: Pierce SuperSignal Femto Maximum sensitivity substrate Cat #37075

Assay Protocol.

A 96 well white polypropylene plate (Coming Cat #CLS3355) was pre-blocked by placing 100 μL of Assay Buffer into each well, incubated overnight then washed three times with wash buffer. 50 μL of each TNF-α APA-Co bead solution was added to every well of 2 columns. The plate was then washed once with wash buffer using the Bio-Tek Elx-405 Magna magnetic plate washer. 100 μL of each TNF-α Antigen Standard was added to the appropriate microtitre wells. The Antigen was incubated with the bead for 1 hour on a plate shaker setting 500 rpm, followed by a 3× wash cycle using Bio-Tek Elx-405 Magna magnetic plate washer. 100 μL of the Anti-TNF Biotinylated Detection Antibody solution was added to the appropriate microtitre wells. The plate was shaken at 500 rpm on a plate shaker for 1 hour at room temperature followed by a 3× wash cycle using Bio-Tek Elx-405 Magna magnetic plate washer. The Strepatvidin-HRP was added to the appropriate microtitre wells and incubated on plate shaker setting 500 rpm for 30 minutes. A final wash cycle of 9× washes was performed using the Bio-Tek Elx-405 Magna magnetic plate washer. 100 μL of the Chemiluminescent substrate was added each well and the plate was incubated on board the BMG Fluostar plate reader at 37 C for 30 minutes. After the 30 minute incubation on board the instrument, the plate was read in Luminescence mode at 37 C.

### RESULTS FROM EXAMPLE 5

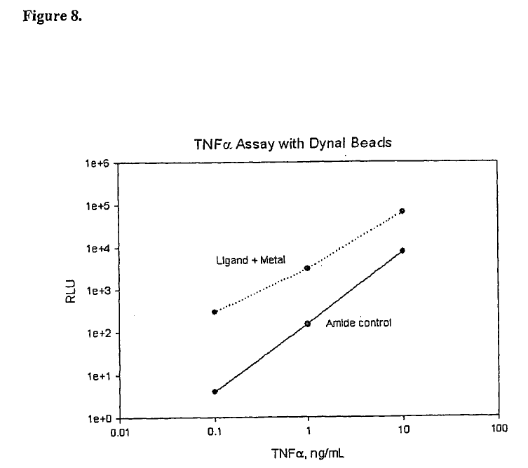

As shown in FIG. 8, synergistic binding gave superior assay sensitivity indicating far more functional antibodies were immobilized on the surface as compared to conventional amide coupling methods.

1. A method of binding a target molecule from an analyte containing the target molecule in admixture with at least one non-target molecule, which method comprises exposing the analyte to a substrate comprising first and second surface binding components,
wherein the first surface binding component has specific binding affinity with respect to the target molecule but is independently unable to provide effective binding of the target molecule to the substrate,
wherein the second surface binding component has non-specific binding affinity with respect to the target molecule and is independently unable to provide effective specific binding of the target molecule to the substrate, and
wherein the target molecule is immobilized on the substrate by the combined binding effect of the first and second surface binding components.

2. A method according to claim 1, wherein the surface binding components are dimensionally smaller than the target molecule with which they can interact.

3. A method according to claim 1, wherein the surface binding components are non-biological in nature,

4. A method according to claim 1, which comprises the use of a plurality of different surface binding components having the binding characteristics as per the first surface binding component and/or the use of a plurality of (different) surface binding components having the characteristics of the second surface binding component.

5. A method according to claim 1, wherein the target molecule is a protein, polypeptide or polynucleotide.

6. A method according to claim 5, wherein the target molecule is an antibody.

7. A method according to claim 1, wherein the target molecule is a pathogen.

8. A method according to claim 1, wherein the interaction between at least one of the first and second surface binding components and the target molecule is thermodynamically reversible so that binding of a target molecule to a substrate may be manipulated.

9. A method according to claim 1, wherein the first and/or second surface binding component is a metal complex.

10. A method according to claim 9, wherein the metal is selected from scandium, titanium, vanadium, chromium, ruthenium, manganese, palladium, iron, cobalt, nickel, copper, molybdenum and zinc.

11. A method according to claim 9, wherein the metal complex comprises a salt moiety selected from chloride, acetate, bromide, nitrate, perchlorate, alum and sulphate.

12. A method according to claim 1, wherein the substrate inherently has appropriate surface functionality that provides at least one of the first and second surface binding components, or provides binding sites to which one or more types of surface binding components may be attached.

13. A method according to claim 1, in which the first and second surface binding components are attached to a structural template.

14. A method according to claim 13, wherein the structural template is, or includes as an integral part thereof, one of the first and second binding components.

15. A method according to claim 13, wherein the structural template is a heterocyclic compound with some affinity and, possibly, selectivity for a target molecule.

16. A method according to claim 15, wherein the second binding component is a metal complex that is able to chelate to the heterocyclic compound.

17. A method according to claim 15, wherein the second binding component is a metal complex that can chelate to a metal chelating ligand that is coupled to the heterocyclic compound.

18. A method according to claim 15, wherein the heterocyclic compound is a triazine.

19. A method of increasing the binding effect of a substrate with respect to a target molecule, the substrate comprising a first surface binding component that has specific binding affinity with respect to the target molecule but that is independently unable to provide effective binding of the target molecule to the substrate, which method comprises providing on the substrate a second surface binding component that has non-specific binding affinity with respect to the target molecule and that is independently unable to provide effective specific binding of the target molecule to the substrate, such that the combined binding effect of the first and second surface binding components results in binding of the target molecule to the substrate.

20. A method of increasing the binding effect of a substrate with respect to a target molecule, the substrate comprising a first surface binding component that has non-specific binding affinity with respect to the target molecule and that is independently unable to provide effective binding of the target molecule to the substrate, which method comprises providing on the substrate a second surface binding component that has specific binding affinity with respect to the target molecule but that is independently unable to provide effective specific binding of the target molecule to the substrate, such that the combined binding effect of the first and second surface binding components results in binding of the target molecule to the substrate.

21. A surface capture agent suitable for extracting a target molecule from an analyte, the surface capture agent comprising first and second surface binding components,
wherein the first surface binding component has specific binding affinity with respect to the target molecule but is independently unable to provide effective binding of the target molecule to the substrate,
wherein in use the second surface binding component has non-specific binding affinity with respect to the target molecule and is independently unable to provide effective specific binding of the target molecule to the substrate, and
wherein the target molecule is immobilized on the substrate by the combined binding effect of the first and second surface binding components.

22. A substrate for immobilizing a target molecule, the substrate comprising first and second surface binding components,
wherein the first surface binding component has specific binding affinity with respect to the target molecule but is independently unable to provide effective binding of the target molecule to the substrate,
wherein the second surface binding component has non-specific binding affinity with respect to the target molecule and is independently unable to provide effective specific binding of the target molecule to the substrate, and
wherein in use the target molecule is immobilized on the substrate by the combined binding effect of the first and second surface binding components.

23. Use of a substrate as claimed in claim 22 in an assay to immobilize a target molecule.

24. A method of designing a capture agent, which comprises identifying first and second surface binding components that are capable of achieving a desired binding interaction between a substrate and a target molecule,
wherein the first surface binding component has specific binding affinity with respect to the target molecule but is independently unable to provide effective binding of the target molecule to the substrate,
wherein the second surface binding component has non-specific binding affinity with respect to the target molecule and is independently unable to provide effective specific binding of the target molecule to the substrate, and
wherein in use the target molecule is immobilized on the substrate by the combined binding effect of the first and second surface binding components.
